(19) United States
(12) Patent Application Publication
(10) Pub. No.: US 2006/0182828 A1
(43) Pub. Date: Aug. 17, 2006

(54) METHODS OF REDUCING LIPID PEROXIDATION AND ACHIEVING RELATED HEALTH BENEFITS BY THE ADMINISTRATION OF TOCOPHEROL AND SESAME LIGNANS

(76) Inventor: John S. Colman, Fort Lauderdale, FL (US)

Correspondence Address: NANCY LORD JOHNSON, LTD., 1970 N. LESLIE RD., NO. 204, PAHRUMP, NV, 89060, US

(21) Appl. No.: 10/906,395

(22) Filed: Feb. 17, 2005

(51) Int. Cl. A61K 31/355; A61K 36/185

(52) U.S. Cl. 424/776; 514/458

(57) ABSTRACT

This invention relates to methods to reduce or lipid peroxidation and diminish free radical damage in a human, resulting in reduced blood levels of dityrosine, isoprostane, and placental growth factor thus preferably preventing or reducing cardiovascular disease by the in humans administration of compositions comprised of sesame lignans and ‘gamma tocopherol to humans.

This application claims priority based on Provisional Application No. 60/61827 which is incorporated by reference in its entirety.

### FIELD OF INVENTION

This invention relates to methods that reduce or lipid peroxidation and diminish free radical damage in a human, resulting in reduced blood levels of dityrosine, isoprostane, and placental-like growth factor thus preferably preventing or reducing cardiovascular disease in humans.

### BACKGROUND OF INVENTION

Atherosclerotic Disease

The present invention relates generally to compositions and methods for treating atherosclerosis; more particularly, it relates to methods and compositions for treating or preventing atherosclerosis whereby the many and varied problems associated with the disease can be prevented, arrested, substantially alleviated or cured.

In the United States and Western Europe, cardiovascular disease and its associated maladies, dysfunctions and complications are a principal cause of disability and the chief cause of death. One specific entity significantly contributing to this pathophysiologic process is atherosclerosis, which has been generally recognized as the leading health care problem both with respect to mortality and health care costs. The American Heart Association estimates that cardiovascular disease accounts for more than 930,000 deaths per year—approximately 38.5% of all deaths in 2001. Eyre, H, et al, 109(25) CIRCULATION 3244-55 (2004). The medical costs associated with coronary heart disease are estimated at $95 billion dollars a year. Gonzalez & Kannewurf, 55 (19) AMERICAN JOURNAL OF HEALTH-SYSTEM PHARMACY S4-7 (Supp. 1, 1998).

Atherosclerosis is a disease characterized by the deposition of fatty substances, primarily cholesterol, and subsequent fibrosis in the arterial intima, resulting in plaque deposition on the inner surface of the arterial wall and degenerative changes within it. The ubiquitous arterial fatty plaque is the earliest lesion of atherosclerosis and is a grossly flat, lipid-rich atheroma consisting of macrophages and smooth muscle fibers. The fibrous plaque of the various forms of advanced atherosclerosis has increased intimal smooth muscle cells surrounded by a connective tissue matrix and variable amounts of intracellular and extracellular lipid. At the luminal surface of the artery, a dense fibrous cap of smooth muscle or connective tissue usually covers this plaque or lesion. Beneath the fibrous cap, the lesions are highly cellular consisting of macrophages, other leukocytes and smooth muscle cells. Deep in this cell-rich region may be areas of cholesterol crystals, necrotic debris and calcification.

If allowed to progress, the disease can cause narrowing and obstruction of the lumen of the artery, diminished or occluded blood flow and, consequently, ischemia or infarction of the predominantly affected organ or anatomical part such as the brain, heart, intestine or extremities. The result can be significant loss of function, loss of cellular substance, emergency medical and/or surgical procedures, and significant disability or death. Alternatively, the arterial wall can be severely weakened by the infiltration of the muscular layer with cholesterol, inflammatory leukocytes, connective tissue and calcium, resulting in soft and/or brittle areas which can become segmentally dilated (aneurysmal) and rupture or crack leading to organ, limb or even life-threatening hemorrhage.

Once the disease has progressed to the stage of significant persistent symptoms and compromised function, the next treatment step has conventionally been artery bypass grafting to repair or replace the damaged artery. While coronary artery bypass has become one of the more common major cardiovascular surgical procedures in the United States, surgery clearly is not the solution to the pathologic process because many patients are reluctant to accept its accompanying significant risk of morbidity and mortality. The autogenous veins or arteries used to bypass the disease-impaired arteries undergo atherosclerotic changes postoperatively generally at a faster rate than the original, affected arteries. The Coronary-Artery Surgery Study (CASS) sponsored by the National Heart, Lung and Blood Institute (NHLBI) concluded that certain subsets of patients do not gain any overall statistical benefit from bypass surgery in comparison to other medical treatments. Carraciolo, 91(9) CIRCULATION 2335-44 (1995).

As an alternative to coronary bypass surgery, certain medications and procedures are used to treat the results of atherosclerosis. These treatments include chelation with ethylene diamine tetra-acetic acid (EDTA) and percutaneous transluminal coronary angioplasty (PTCA). EDTA treatments, however, are still experimental, unproved and potentially as harmful as they are beneficial. PTCA treatments are invasive, of limited application and success, and occasionally lethal. Highly experimental intra-arterial laser beam plaque vaporization has limited application and requires an open operative approach to affected vessels.

It is now well established that vascular blockage and cardiovascular disorders including myocardial infarction, coronary heart disease, hypertension and hypotension, cerebrovascular disorders including stroke, cerebral thrombosis and memory loss due to stroke; peripheral vascular disease and intestinal infarction are caused by blockage of arteries and arterioles by atherosclerotic plaque. The production of atherosclerotic plaque formation is multi-factorial in its production. Hypercholesterolemia, especially elevated levels of low-density lipoprotein cholesterol (LDL) is an important risk factor for atherosclerosis, arteriosclerosis and associated diseases.

Lipid Peroxidation

In accordance with the present invention, methods and compositions are provided for use in treating atherosclerosis and its associated diseases including cardiovascular disorders, cerebrovascular disorders, peripheral vascular disorders, and intestinal vascular disorders. The compositions of the present invention can be administered prophylactically, so as to inhibit atherogenesis or restenosis, or therapeutically after atherogenesis has been initiated. Thus, for example, a patient who is to undergo balloon angioplasty can have a regimen of the composition administered substantially prior to the balloon angioplasty, preferably at least about a week or substantially longer. Alternatively, in a patient where atherogenesis is suspected, the administration the composition can begin at any time. Administration may be accomplished in any manner known to those skilled in the art, including peroral, liposomal, inhalation, sublingual, rectal (e.g., suppositories), or through an oral spray or dermal patch.

LDL oxidation is now believed to be the primary initiating event in human atherosclerosis and is a target for disease slowing interventions. Steinberg, D, et al, 320(14) N ENGL J MED. 915-24 (1989). It is believed to be controlled or prevented by antioxidants such as tocopherols. The circulating chylomicrons are protein particles that transport cholesterol reversibly to and from the liver. The chylomicrons are divided into four groups: LDL's, HDL'S, VLDL'S and IDL'S. Several oxidants such as peroxynitrite radical, iron/ascorbate and copper/ascorbate present in the blood and serum oxidize LDL. Esterbauer, H, et al, 13(4) FREE RADIC BIOL MED 341-90 (1992). Once LDL is oxidatively modified, it is no longer recognized by LDL receptors but by the scavenger receptors on macrophages which leads to the formation of foam cells. Miura, S, Watanabe, J 18(1) BIOL PHARM BULL 1-4 (1995). Sesame oil is highly resistant to oxidative deterioration and this is likely due to the presence of endogenous antioxidants that include its lignans. Kang, et al, 66(2) LIFE SCIENCES 161-171 (2000).

The earliest recognized gross lesion in atherogenesis is the fatty streak, characterized by an accumulation of foam cells loaded with cholesteryl esters just beneath the vascular endothelium. The LDL receptor in the arteries gives rise to foam cells and fatty streaks, the earliest lesion in atherosclerosis, but there is also a receptor-independent mechanism for their formation. Esterbauer et al, 38 ADV. PHARMACOL. 425-56 (1 997); Esterbauer, 2 NUTR. METAB. CARDIOVASC. DIS. 55-7 (1992). This has been demonstrated by the development of lesions rich in macrophage-derived foam cells, even in patients and animals deficient in LDL receptors, and the failure to produce foam cells from normal monocytes and monocyte derived macrophages incubated with LDL. This led researchers to explore the possibility of a post-secretory modification of LDL before it is taken up into foam cells by a specific receptor. Steinberg, 320(1 4) NEW ENG. JL. MED. 915-924 (1989).

Researchers have shown that when LDL was incubated with cultured endothelial cells it underwent a striking series of physical and chemical changes and was taken up by cultured macrophages 10 times more rapidly than native LDL. Id., at 916. At any given level of hypercholesterolemia there is considerable variation in clinical disease. Postsecretory modifications in the structure of lipoproteins appear to effect their atherogenic potential. Steinberg, Ibid, at 915. It is not only the elevated levels of LDL cholesterol that are important, but also its oxidation, that leads to atherosclerosis, thus antioxidants are believed to reduce the risk of atherosclerotic disease. Mortensen, 18 MOLECULAR ASPECTS OF MEDICINE s137-s144, (Supp. 1997). Peroxidation of polyunsaturated fatty acids in the LDL lipids is the common initiating factor of the changes, and the cytotoxicity of oxidized LDL has been proven by several research groups, and may lead to the denudation of the benign fatty-streak lesion into the atheromatous plaque. Steinberg, supra, at 918.

Researchers believe that the oxidation of LDL within the arterial wall itself is most important. Ocana, 321(17) NEW ENG. J. MEDICINE 1196-1197 (1989). Auto-antibodies to MDL-LDL were seen at significantly higher titers in men with atherosclerosis than in normal controls, and in a greater proportion of smokers, those with higher LDL cholesterol, and those with higher serum levels of copper in the case group. Salonen, 339 LANCET 883-887 (1992).

Researchers also have studied the effects of incubation of LDL with macrophages and found that in that environment LDL is oxidized and recognized and taken up by the acetyl LDL or scavenger receptor in the same cell. Alpha-tocopherol, butylated hydroxytoluene (BHT) and Probucol block this process. Parthasarathy, 6(5) ARTERIOSCLEROSIS 505-10 (1986). Treatment with Probucol, a potent anti-oxidant, significantly lowered the rate of development of fatty streak lesions in hyperlipidemic rabbits, although the plasma cholesterol level was not lower than in lovastatin-treated animals. Carew, Schwenke & Steinberg, 84 PROC. NAT'L ACAD. SCI. USA 7725-7729 (1987). Similar results have been demonstrated in cultures of LDL with endothelial cells. Steinbrecher, 81 PROC. NAT'L ACAD. SCI. 3883-3887 (1984). Monocytes and neutrophils, when incubated with LDL, oxidize LDL and render it toxic. Cathcart, Morel & Chisolm, 38 J. LEUKOCYTE BIOLOGY 341-350 (1985).

### SUMMARY OF INVENTION

This inventor has discovered that sesame lignans, at low doses, most specifically at about 100 mg, with gamma-tocopherol at about 420 mg produce a reduction in lipid peroxidation that is demonstrated with reliable markers, PLGF-1, dityrosine, and isoprostane. The combined administration of natural or synthetic tocopherol, most specifically gamma tocopherol, in combination with sesame lignans, most specifically episesamin, reduces lipid peroxidation and diminishes free radical damage in a human. The composition is comprised of one or more natural tocopherols, synthetic tocopherol isomer, tocopherol analogue, or pharmaceutically acceptable salt or ester of a tocopherol, and one or more sesame lignans. The reduction in lipid peroxidation is measurable through the use of proven markers of lipid peroxidation, blood dityrosine levels, blood isoprostane levels, and blood Placental-Like Growth Factor (PLGF-1).

In accordance with the present invention, methods and compositions are provided to treat a human who suffers from one or more conditions related to oxidative stress including atherosclerosis, premature aging, Alzheimer's disease, stroke, toxic hepatitis, viral hepatitis, peripheral vascular insufficiency, renal disease, and hyperglycemia. Or, it may be used in a healthy human who seeks to optimize his or her health.

The invention discloses the optimal doses to be used to achieve the reduction in lipid peroxidation that may be demonstrated by the markers of blood dityrosine, isoprostane, and PLGF-1. The optimal ranges are gamma tocopherol in an amount of about 100 IU to about 2000 IU and one or more sesame lignans in an amount of about 10 mg to about 500 mg total; more specifically gamma tocopherol in an amount of about 200 IU to about 1000 IU and one or more sesame lignans in an amount of about 20 mg to about 200 mg total; and most specifically gamma tocopherol in an amount of about 420 IU and one or more sesame lignans in an amount of about 100 mg total. At these doses, the method is effective to reduce lipid peroxidation; reduce the oxidation of HDL and LDL in a human; increase levels of gamma tocopherol in human; reduce chylomicron oxidation and diminish free radical damage to a human.

### DETAILED DESCRIPTION OF INVENTION

Vitamin E

Vitamin E, a potent antioxidant, has been shown to reduce the extent of atherosclerosis in several animal models and studies have shown that Vitamin E can be protective against the disease. Pryor 28(1) FREE RADICAL BIOLOGY & MEDICINE 141-64 (2000). The development of the fatty streak lesion may be based upon two factors: the presence of elevated plasma LDL and its oxidative modification within the artery wall. Steinberg, supra, at 91 9. LDL particles in whole plasma contain the antioxidant compounds vitamin E and .beta.-carotenes and the plasma itself contains antioxidants that protect the LDL for a relatively short time. Under pro-oxidant conditions, the vitamin E and .beta.-carotene are destroyed before the fatty acids undergo peroxidation. Id., at 921. It is likely that decreases in vitamin E and beta-carotene are early events reflecting the initial stages of lipid peroxidation. Witztum & Steinberg, 88(6) J. CLINICAL INVESTIGATION 1785-1792 (1991).

It has now been generally accepted that LDL cholesterol becomes harmful only in its oxidized form. Schwartz, C J et al. 14(2 Suppl 1) CLIN. CARDIOL. I1-16 (1991); Jialal, I, Grundy, S M, 87(2) J. CLIN INVEST. 597-601 (1991). Native LDL consists of phospholipids, triglycerides, cholesterol, both free and as an ester, fatty acids (50% of which is polyunsaturated), proteins and lipophilic antioxidants that protect the polyunsaturated fatty acids (PUFA) in cholesterol against free radical attack and oxidation. The first step in the oxidation of cholesterol is the production of free radicals, which are generally induced by oxidative stress. These radicals act to deplete lipids of their natural antioxidants, such as vitamin E and carotinoids, and are also highly reactive against proteins, DNA, polyunsaturated fatty acids (PUFA) and lipids. Once the natural antioxidants are depleted, the free radicals move to oxidize unprotected LDL. The oxidized cholesterol molecule is recognized by scavenger receptors and internalized by macrophages in the form of lipid laden foam cells, the first step in the formation of artherosclerotic plaque Esterbauer et al, 38 ADV. PHARMACOL. 425-56 (1997); Esterbauer, 2 NUTR. METAB. CARDIOVASC. DIS. 55-7 (1992). Oxidative stress may occur when formation of reactive oxygen species increases, scavenging of reactive oxygen species or repair of oxidatively damaged macromolecules decreases, or both. Thus, factors such as exposure to environmental pollutants and pesticides can instigate the generation of oxysterols internally.

Vitamin E has been studied in depth for its effects on cardiovascular disease. For example, studies have shown that supplementation with just 30 IU to 100 IU of vitamin E lowers the risk of heart disease by 41%. Murray and Pizzorno, Encyclopedia of Natural Medicine 91 (2nd ed., 1997) (1998). Another study showed that supplementation with 100 IU of vitamin E results in reduced progression of coronary artery disease (Id.). Despite these earlier promising results, more recent findings suggest that vitamin E has no effect on foam cell production, although supplementation with vitamin E increases the levels of vitamin E in cells such as macrophages. The same study concluded that there is a direct correlation between foam cell production and depletion of cellular vitamin E, though this does not correlate with the amount of cell lysis by oxidized LDL. Asmis et al., 20 ARTERIOSCLER THROMB VASC BIOL 2078-86 (2000).

Sesame

Sesame is one of the largest and most important oil seed crops in the world, used as a wholesome food for humans and a source of edible oil. The seeds and its oil have been considered a health food in Japan and China to prevent aging, but the scientific aspects of its chemical composition and biochemical effects have only been explored intensively in the last decade. Sesame lignans are the one percent solid portion of sesame oil and are responsible for all the beneficial effects of sesame.

Sesame seed and sesame oil contain several lignans including sesamin, episesamin, sesaminol, sesamol, sesamolin and sesamolinol. The crude mixture of sesame lignans is believed be highly synergistic and many studies use total sesame lignans or total defatted sesame flour for their testing. Kang, M et al, 129 J NUTR 1885-90 (1999).

Sesame and its lignans have a broad range of applications in human health. They have been shown to increase human tissue and serum alpha tocopherol, gamma tocopherol and tocotrienol levels dramatically, Yamashita, K., 30(11) LIPIDS 1019-28 (1995); Sontag, T. J., 277(28) J Biol Chem 25290-6 (2002), Sontag, T., Parker, S, 277 (28) J. BIOL CHEM. 25290-96; Ikeda, S, Yoyoshima, K., and Yamashita, K, 131 J NUTR: 2892-2897 (2001); increase the anti-inflammatory index of fish oils, potently block LDL cholesterol oxidation, Kang, M H, Naito, M, 66(2) LIFE SCI 161-71 (2000), Miura, S, Watanabe, J 18(1) BIOL PHARM BULL 1-4 (1995); inhibit lipid peroxidation, Kang, M-H, et al, 66(2) LIFE SCIENCES 161-71 (2000); lower total and LDL serum cholesterol in humans, Hirata, F, et al, 122 ATHEROSCLEROSIS 135-6 (1996); Nakabayashi, A, et al, 65(3) INTJ VITAM RES 162-8 (1995); protect against alcohol damage, Akimoto, et al, 37 ANN NUTR METAB 218-224 (1993); protect against multiple organ failure Hsu D Z, Liu M Y, 30(8) CRIT CARE MED. 1 859-62 (2002); and act as a mild antihypertensive in DOCA-salt rats, Nakano, D, 25(9) BIOL PHARM BULL 1247-9 (2002) and stroke-prone spontaneously hypertensive rats, Matusmura, Y, et al, 21(5) BIOL PHARM BULL 469-73 (1988).

The ability of sesamol to reduce the synthesis of the coenzyme, NADPH, that makes it attractive for use in studying the effect of oxidants on tumor and vascular endothelial cells. Jacklin A, et al, 1010 ANN NY ACAD SC 374-80 (2003). It also offers protection against a wide variety of carcinogens, and has been shown to reduce the formation of mouse skin papillomas induced with 12-0-tetradecanoylphorbol 1 acetate by 50%, comparable to the 60% reduction with resveratrol. Kapadia, G et al, 45(6) PHARMACOL RES 499-505 (2002); Hibasami, H, et al, 7(6) ONCOL REP 1213-6 (2000). In a study of human lymphoid leukemia cells, morphological change showing apoptotic bodies were observed in cells treated with sesamin and episesamin. The fragmentations by sesamin and episesamin of DNA to oligonucleosomal-sized fragments that are characteristics of apoptosis were observed to be concentration-dependent. Miyahara, Y, et al, 7(5) INT J MOL MED 485-8 (2001). Growth inhibition of the cells was also noted. Miyahara, Y, et al, 7(4) INT J MOL MED 369-71 (2001), preventing senescence, and slowing aging.

Sesame Lignans, Vitamin E and Tocotrienols

In human and animal studies, administration of sesame lignans increases tissue and serum levels of all vitamin E analogs—especially alpha tocopherol, gamma tocopherol and tocotrienols. This has been confirmed many times in both in vitro and in vivo studies with humans and animals. It is especially important to note that sesame lignans elevate gamma tocopherol levels quite dramatically, because gamma tocopherol, but not alpha tocopherol, quenches reactive nitrogen species, such as peroxynitrite radical, which we now know plays a major role in the development of cardiovascular diseases and atherosclerosis. Kontush, A., et al, 144(1) ATHEROSCLEROSIS 117-22 (1999).

The difficulty of raising alpha- and gamma-tocopherols, even with the addition of sesame lignans has been noted by several researchers. Ikeda, S, Yoyoshima, K., and Yamashita, K, 131 J NUTR 2892-2897 (2001). Ikeda reports that tocotrienols were not detected in any tissues or plasma of the deficient, alpha-tocopherol plus gamma-tocopherol, and alpha-tocopherol plus sesame groups. Two concurrent forces make raising gamma tocopherol levels in human tissues and serum difficult. Only alpha-tocopherol carrier protein transports vitamin E from the liver to the tissues and it is highly selective in transporting only alpha tocopherol in a 20 to one ratio to other tocopherols and little gamma tocopherol ends up in the cell membranes. Most gamma tocopherol is excreted through the bile without ever combining with the transporter protein because gamma tocopherol has been found to bind very weakly to the tocopherol carrier protein. Yamashita K, 30 LIPIDS 1019-28 (1995). The liver P-450 enzyme that metabolizes tocopherol analogs, tocopherol-omega-hydroxylase, has a markedly higher catalytic activity for gamma-tocopherol than alpha-tocopherol. Sesamin potently inhibits the activity of hepatic tocopherol-omega-hydroxylase, which is a physiologically important mechanism in the regulation of vitamin E status. Sontag, T J, and Parker, R S, 277(28) JL. BIOL. CHEM. 25290-25296 (2002). A further study showed that sesame inhibits tocopherol gamma-hydroxylase, or CYP-3A, the specific enzyme that breaks down tocopherols and tocotrienols. Parker R S, Sontag T J, Swanson J E, 277(3) BIOCHEM BIOPHYS RES COMMUN 531-4 (2000). This may explain why taking gamma tocopherol supplements contemporaneously with vitamin E results in the detection of low serum and tissue levels of gamma-tocopherol being detected. Kayden, M J, Traber, M G, 34(3) J LIPID RES 343-58 (1993). This inventor has identified specific dosage ranges that produce increased tissue and plasma levels of alpha- and gamma-tocopherol as measured by the specific tests used in this invention.

Several studies have shown an increased prevalence for coronary heart disease in patients with lower plasma gamma-tocopherol concentrations as opposed to controls. Kushi, et al, 334 New Eng JI Med 1156-1162 (1996). Low plasma concentrations of gamma-tocopherol has been suggested as a more sensitive sensitive risk index for atherosclerosis than measuring for low alpha-tocopherol concentrations. Id. In postmenopausal women, intake of foods high in gamma-tocopherol was inversely related to the risk of death from coronary heart disease, but this same risk did not diminish in women supplementing with vitamin E. Ibid. In a study of Swedish women, supplementing with sesame oil, and no additional tocopherols, raised gamma-tocopherol levels 41.7%. Lemcke-Norojarvi, M, et al, 131(4) J NUTR 1195-201 (2001). In that study, unlike the present invention, the sesame was provided as oil in buns, three of which were consumed per day for a total dose of 22.5 g/day, without additional gamma-tocopherol. This inventor has discovered a method for increasing gamma-tocopherol by administering it with sesame lignans in much lower doses that will be easier to consume and will not increase the daily consumption of calories required by Lemcke's teachings.

In animal studies, supplementing with gamma-tocopherol alone resulted in only small concentrations of gamma-tocopherol in the plasma and liver of rats and the animals had greater lipid peroxidation rates than alpha-tocopherol-fed animals. In sharp contrast, rats fed sesame lignans had high concentrations of gamma-tocopherol found in plasma and liver and greatly reduced lipid peroxidation rates. Yamashita, K et al, 122 J NUTR 2440-6 (1992). Lipid peroxidation rates in blood plasma in the sesame/gamma-tocopherol group were 50% lower, and liver lipid peroxidations rates were 30% lower, than the animals fed only gamma-tocopherol. Yamashita, Y 30(11) LIPIDS 1019-28 (1995). In a study of alpha- and gamma-tocotrienol levels in the skin and adipose tissue of rats fed tocotrienol-rich fraction (TRF) from palm oil with either gamma-tocopherol or sesame, and alpha-tocopherol with or without sesame, the alpha- and gamma-tocotrienol concentrations in perirenal adipose tissue, epididymal fat and skin of the TRF+sesame groups were significantly higher (P<0.05) than those of the TRF+gamma-tocotrienol group. This study measured the levels of tocotrienols, but did not measure the effect of the tocotrienols on the peroxidation of lipids.

Comparisons between human and animal studies shows that supplementing with sesame or its lignans produces similar effects in raising all tocopherol and tocotrienol levels. In one study, liver, brain, kidney and serum levels of gamma tocopherol in rats were measured after administration of gamma-tocopherol alone or being given gamma-tocopherol and sesame. The gamma-tocopherol-only fed group increased liver, kidney, brain and serum levels of gamma tocopherol only 3 nmol per gram in tissues and 3 micromoles per liter in serum. When fed sesame and gamma tocopherol together, the rats had gamma tocopherol levels of 25-30 nmol per gram in tissue and 30 nmol per liter, an increase of between 833 to 1,000 percent. Ikeda S, Tohyama T, Yamashita K, 132(5) J Nutr 961-6 (2002). The urinary excretion of gamma-CEHC, the metabolite of gamma tocopherol dropped 50% in the sesame-supplemented rats. Other tests have shown that adding sesame seed to a rat diet increased vitamin E serum levels 40%, and gamma tocopherol levels 800%, while lowering levels of liver thiobarbituric reactive substance (TBARS), a marker for DNA damage, by 270% Id., Kang, et al, 66(2) LIFE SCIENCES 161-171 (2000).

To demonstrate the effect of adding sesame lignans to a tocotrienol-rich diet, Japanese researchers added sesame to the diet of rats fed palm oil tocotrienols. Sesame and tocotrienol supplementation on alternate days resulted in significantly higher levels of alpha tocotrienol, alpha tocopherol, and gamma tocopherol levels in serum, kidney and various other tissues. Yamashita, K, et al, 37 LIPIDS 351-8 (2002).

A standardized test has been developed that measures how much LDL or HDL is oxidized in a 100 minute time period when exposed to copper/iron-ascorbate in the presence of various antioxidants and the lag time is recorded. Esterbauer, H, et al, 13 (4) FREE RADIC BIOL MED 341-90 (1992). Until 1995, BHT and tetrahydrocurcumin were among the most potent inhibitors of Cu-ascorbate induced lipid peroxidation, but sesaminol, the most prevelant sesame lignan, inhibited the lag time of LDL oxidation by 510 minutes, 567% longer than tetrahydrocurcumin. The chart below compares the lag time of sesaminol with other antioxidants. Kang, et al, 66(2) LIFE SCIENCES 161-171 (2000).

Until recently, the standard marker for oxidative stress was the formation of Thiobarbituric Acid Reactive Substances (TBARS). In another study probing the exact mechanisms of LDL oxidation protection of sesame lignans, it was found that sesaminol inhibited TBARS formation by 82.8% at 0.1 micromolar concentration and 100% protection at one micromolar concentration, both being extremely low concentrations that can easily be reached by dietary supplementation. Sesaminol also inhibited the formation of 4-hydroxy-2-nonenal (4-HNE) and malondialdehyde (MDA), the two most damaging aldehydes in biology to 17 and 13% of control values, while probucal and alpha-tocopherol had absolutely no effect in blocking these two toxic metabolites. Kang, et al, 66(2) LIFE SCIENCES 161-171 (2000); Kang, M et al, 129 J NUTR 1885-90 (1999). The authors concluded that sesaminol strongly inhibited the lipid peroxidation of LDL in a concentration-dependent manner, with the IC50 value calculated to be 36.0±10.0 nM. Kang, Ibid. In another study, the addition of sesaminol significantly suppressed liver TBARS in rats fed 20 mg/kg and 50 mg/kg of palm oil to nearly the same as those seen in the 50 mg/kg alpha-tocopherol control diet. Yamashita et al, 37 LIPIDS 351-8 (2002). In chromatographic studies, the time-dependent formation of TBARS in 2,2′-azobis(2,4-dimethylvaleronitrile)(AMVN)-induced low-density lipoprotein (LDL) peroxidation was monitored by TBARS formation, and a statistically significant difference (P<0.05) was observed. Kang, M, Katsuzaki and Osawa, T, 33(10) LIPIDS 1031-6 (1998).

Sesame lignans have been noted for their improvements in lipid profiles in both humans and experimental animals. Sesame lowers triglycerides by increasing fatty acid metabolism in the liver, but does not have a significant effect on cholesterol. Hirata, F, et al, 122(1) ATHEROSCLEROSIS 135-36 (1996). However, sesame lignans lowers total cholesterol in hypercholesteremic rabbits and protects them against oxidative stress. Kang, M et al, 129 J NUTR 1885-90 (1999).

Among natural compounds, it is well demonstrated that n-3 polyunsaturated fatty acids such as alpha-linolenic, eicosapentaenoic and docosahexaenoic acids increase fatty acid oxidation in the liver as does sesamin. However, diets containing at least 3-10% of these fatty acids need to be consumed to have a meaningful physiological effect in rats. These authors measured gene exprssion and activity of enzymes involved in fatty acid syntheses in rats fed no sesamin, and sesamin at 0.2% and 0.4% of diet. They showed that dietary sesamin dose-dependently decreased the expression of sterol regulatory element binding protein-1 (SREBP-1) m RNA level, which was one-half that of rats fed a sesamin-free diet in the 0.4% sesamin diet group. Ide, T., et al, 1534 BIOCHIMICA ET BIOPHYSICA ACTA 1-13 (2001).

Hypercholesterolemic men first given 32.4 then 64.8 mg of sesamin for a total of 8 weeks demonstrated an mean reduction of total cholesterol from 272.0 to 248.3 and LDL cholesterol from 187.1 to 156.4, but no changes in triglycerides or HDL cholesterol, although apolipoprotein B dropped from 188.8 to 168.5. No changes were observed in the placebo group. Hirata, F, et al, 122 ATHEROSCLEROSIS 135-6 (1996).

Other animal experimental models have reported a reduction of cholesterol biosynthesis rate limiting enzyme HMG-Coenzme-A-reductase, a reduction of intestinal cholesterol re-absorption, increases in cholesterol bile excretion, and inhibition of acyl-Co-A-cholesterol acyltransferase, all contributing to an improved lipid profile. Nakabayashi, A, et al, 65(3) INTJ VITAM RES 162-8 (1995). Vitamin E and sesamin was added to the diets of rats lowered LDL and VLDL cholesterol levels and increased HDL cholesterol levels significantly, favorably improving the atherogenic index.

Sesame lignans have a very mild antihypertensive effect that has been observed in animals but not noted or commented on in humans. Nakano, D, ltoh, C, 25(9) BIOL. PHARM BULL 1247-9 (2002). Matsumura, Y, et al, 21(5) BIOL PHARM BULL 469-73 (1998) In both normotensive and hypertensive stroke-prone rats sesame exerts a mild antihypertensive effect by inhibiting vascular superoxide production. In rats fed high salt diets, sesame prevented the development of hypertension and development of histological renal damage such as thickening of the tunica intima and fibrinoid degeneration of the arterial wall. Matsumura, Y, et al 18(7) Biol Pharm Bull. 1016-9 (1995). This may have implications in humans because of the high salt content in the American diet.

Sesame lignans and sesamin inhibit the enzyme delta-5-desaturase, a chain-shortening enzyme that takes long chain lipids like EPA-6's and shortens them to EPA-3'S. Shimizu, S, et al, 26(7) LIPIDS 512-6 (1991). This is important because one EPA-6 in fish oil is highly anti-inflammatory DGLA, or dihomo-gamma-linolenic acid. This EPA-6 lipid is broken down quickly by mammals to an inactive EPA-3, that it can exert little or no antiinflammatory activity in vivo. Yet when sesame lignans are combined with fish oil, DGLA tissue levels rise dramatically and the inflammatory index of the fish oil also greatly improves. Utsunomiya T, 72(3) AM J CLIN NUTR 804-8 (2000). DGLA also works its antinflammatory effects, in part, by displacing arachidonic acid and thereby decreasing the formation of pro-inflammatory 2-series prostaglandins. Delta-5-desaturase converts DGLA to the highly inflammatory substance arachidonic acid, and this effect is also blocked by sesame lignans.

This anti-inflammatory effect was noted in animals fed linseed oil and sesame lignans. Linseed oil is also a source of DGLA, but like fish oil, its DGLA breaks down rapidly without added sesame. Linseed plus sesame lowered TNF-alpha plasma concentrations in rats (199 ng/L vs. 488 ng./L) and reduced PGE-2 blood levels to 197 ng/L from 335 ng./L. The authors concluded that a significant accumulation of DGLA in the tissues of rats fed sesame was associated with a decline in inflammation caused by a reduction of TNF-alpha and PGE-2 levels and by a synergistic effect on the anti-inflammatory properties of EPA-3's. Fujiyama-Fujiwara Y, et al, 41(2) NUTR SCI VITAMINOL 21 7-25 (1995).

The addition of sesame lignans to fish oil also lowers lipid peroxidation rates caused by the fish oil EPA-3's. An undesirable side effect of supplementing with highly polyunsaturated fats is increased free radical production. Denham Harman was the first researcher to demonstrate the higher degree of polyunsaturated fatty acids in the diets of mice, the higher the cancer rates. Sesame suppresses the formation of lipid peroxides from DHA, which has extremely high oxidative susceptibility but is also one of the main EPA-3's in fish oil that blocks inflammation. DGLA, a polyunsaturated fatty acid, is extremely vulnerable to oxidation and contributes to free radical propagation, yet is very anti-inflammatory. The combination of sesame and DGLA suppresses free radical production by EPA's by blocking lipid peroxidation and blocking the oxidation of DHA-DGLA, thus increasing their levels. by blocking their oxidation. Ikeda, S, et al, 49(4) J NUTR SCI VITAMINOL 270-6 (2003).

Rats fed both sesame lignans and fish oil had higher hepatic fatty acid oxidation rates than those fed sesame with palm oil or safflower oil combinations. This study pointed out that sesame-fish oil combinations work synergistically by increasing the gene expression of peroxisomal fatty acid oxidation enzymes and acyl-CoA oxidase activity in the liver. Umeda-Sawada R, Ogawa M, Igarashi O, 34(6) LIPIDS 633-7 (1999).

Fish oil sesame combinations have a very high anti-inflammatory index and the latest obesity theories are based on the fact that inflammation lowers leptin production which shuts off lipogenesis. McCarty, M F, 57(3) MED HYPOTHESES 324-36 (2001). Sesame rich in lignans results in increased activity of hepatic fatty acid oxidation enzymes including acyl-CoA oxidase, arnitine palmitoyl transferase, 3-hydroxyacyl-CoA dehydrogenase, and 3-ketoacyl-CoA thiolase and lowered serum triacylglycerols. Sirato-Yasumoto, S., et al, 49 J. AGRIC. FOOD CHEM. 2647-2651 (2001). In another study, sesamin and episesamin also significantly increased the peroxisomal palmitoyl Co-A oxidation rate. The extent of the increase was much more exaggerated with episesamin (5.1 fold) than with sesamin (1.6-fold). Alterations in hepatic fatty acid synthesis and oxidation are crucial factors affecting the assembly and secretion of very-low density lipoproteins (VLDL) and hence modify serum lipid levels. Kushiro, M, et al, 13 JL. NUTRIT. BIOCHEM. 289-295 (2002).

A test of this effect was confirmed in rats fed a high DHA diet[ from fish oil] which first lowered vitamin E levels and raised TBARS. Addition of sesame to the diet raised plasma and liver vitamin E levels, increased tissue and plasma DHA, and completely suppressed free radical production from the DHA in tissues and serum. Gu, J Y et al 59 (12) BIOSCI BIOTECHNOL BIOCHEM 2198-202 (1995).

Sesame and Fatty Acid Oxidation

Sesame and its lignans have been shown to possess multiple health benefits either by themselves or in combination with other products where they act in a highly synergistic manner to accentuate the best properties of many supplements. They elevate alpha tocopherol, gamma tocopherol and tocotrienol tissue and serum levels, antioxidants which are strongly correlated with increased longevity in mammals and humans. They increase both antinflammatory DGLA and DHA levels and block free radical lipid peroxidation from fish oil supplementation which will give us a new tool to mitigate inflammation. They are the most powerful inhibitors of LDL oxidation yet discovered, improve the atherogenic profile in humans, and, though some seed lines are more potent than others, are potent stimulators of fatty acid oxidation a key factor in weight loss. Sirato-Yasumoto, et al, 49 J AGRIC FOOD CHEM 2647-2651 (2001).

Numerous studies note that sesame is a potential weight loss agent because it remarkably increases fatty acid oxidation in the liver. Kushiro, et al, 13 J NUTR BIOCHEM 289-295 (2002). Episesamin is a sesame lignan that has an even more powerful effect on hepatic fatty acid oxidation than sesamin. In one study, episesamin stimulated the fatty acid oxidation rate to 1.5 to 14 times that of the control diet, while sesamin only stimulated fatty acid oxidation in the range of 1.3 to 2.8 fold rate of the control diet. Kushiro, et al, 13(5) J NUTR BIOCHEM 289-295 (2002).

Lipid Peroxidation Markers

### Placental-Like Growth Factor

Recent work has established a fundamental role for inflammation in mediating initiation and progression of atherosclerosis and its thrombotic complications. Placental growth factor (PLGF-1) is a vascular endothelial growth factor and has recently been shown to be upregulated in early and advanced atherosclerotic lesions. In a recent study, researchers established a direct correlation between elevated PLGF-1 levels, >27.0 ng/L, and increased risk of cardiovascular events within 30 days (P<0.001). This was a stronger correlate than any other factor studied, including age, hypertension, diabetes, and C reactive protein. PLGF-1 is shown to be a primary inflammatory instigator in animal models of atherosclerosis and arthritis. Heeschen, C, 291(4) JAMA 435-441 (2004(.

PLGF-1, or placental-like growth factor, a member of the vascular endothelial family of growth factors, is an extremely sensitive new test for cardiovascular or atherosclerotic risk in humans recently shown to be upregulated in early and advanced atherosclerotic lesions. Originally identified in the placenta, PLGF-1 stimulates vascular smooth muscle cell growth, recruits macrophages into atherosclerotic lesions, up-regulates production of tumor-necrosis factor and monocyte chemotactic protein-i by macrophages, and stimulates pathological angiogenesis. Inhibition of PLGF-1 by blocking its receptor Fms-like tyrosine kinase and suppressed both atherosclerotic plaque growth and vulnerability via inhibition of inflammatory cell infiltration in an animal model. These data suggest that PLGF-1 may act as a primary inflammatory instigator of atherosclerotic plaque instability. Id.

### Iso-Prostaglandin

The compound, 8-iso-prostaglandin-F-2-alpha is one of a large number of prostanes produced predominantly by free radical catalyzed peroxidation of arachidonic acid. The iso-prostaglandin and its dimer metabolite are elevated in animal models of free radical injury, Alzheimer's disease, smokers and type 2 diabetes and can be measured in urine. This marker of oxidative stress is significantly and two fold higher in smokers than in non-smokers (P<0.02). The authors expect that this measurement will facilitate the monitoring of antioxidants, drugs or dietary manipulations for this marker of peroxidation. Liang, et al, 34(4) FREE RADICAL BIOLOGY & MEDICINE 409-418 (2003).

Iso-prostaglandin serum level is a newer measurement of cell membrane damage that is a much more accurate measure than the older TBARS test, which detected all serum aldehydes.

### Dityrosine

Because o,o′-dityrosine, which appears when hydroxy radical cross-links tyrosine residens, and o-tyrosine, which appears when hydroxyl radical oxidizes protein- bound phenylalanine residens, are stable to acid hydrolysis and thus potentially useful markers for protein oxidation in vivo. Their derivatives have been isolated from urine. In a study of sedentary and exercise trained rats on either a control or antioxidant diet, levels of alpha-tocopherol, beta-carotene, and retinyl ester were higher in those on an the antioxidant diet. Supplementation reduced the levels of protein-bound o,o′-dityrosine in skeletal muscle of both sedentary and exercise trained rats, and also reduced the levels of urinary o,o′-dityrosine in sedentary rats to one-half that of controls (P<0.005). Exercise produced similar reductions. Measurement of plasma levels of isoprostanes has implicated lipid peroxidation as one pathway for oxidative stress in humans. Leeuwenburgh, C, 27 AM. J. PHYSIOL. R 128-R135 (1999).

Dityrosine serum levels is a measure of deep tissue protein oxidation of peroxynitrite radical reacting with tyrosine, one of the amino acids found in all human proteins. It is the amino acid most readily attacked by peroxynitrite radical. Peroxynitrite radical is an extremely powerful free radical that has a very fast rate constant. It is particularly implicated in atherosclerosis and increasingly in Alzheimer's, Parkinson's and other degenerative diseases of aging. Gamma-tocopherol is the only tocopherol isoform that reacts with, or traps, peroxynitrite radical to any appreciable degree.

This inventor has discovered that the remarkable results seen in rodents are reproducible in humans at specific dosages, and has demonstrated that the addition of sesame lignans to gamma tocopherol supplementation will provide clinically meaningful decreases in oxidative stress as measured in recently developed tests.

### EXAMPLE 1

In a clinical study performed on sixteen human volunteers, biomarkers of oxidative stress PLGF-1, dityrosine, and isoprostane, were measured in a gamma-tocopherol only supplemented group and two sesame lignans and gamma-tocopherol combined supplemented groups.

One group of 5 women and 3 men recieved 100 mg sesame lignans with 420 gamma-tocopherol and tocotrienols (100 S), the other, of 6 women and 2 men, received 400 mg sesame lignans with 420 gamma-tocopherol and tocotrienols (400 S), and another received only the 420 gamma-tocopherol and tocotrienols. There were, however, demographic differences and several drop-outs in the gamma-tocopherol only group. One of the subjects, both women, in both the 100 S and 400 S group dropped out for unavailability during the 2004 Florida hurricanes, and another in the 400 S group found that the study medicine did not agree with her. Baselines and post-administration measurements were taken of PLGF-1, dityrosine, and isoprostane.

| Means for: | PLGF-1 | Dityrosine | Isoprotane |
|---|---|---|---|
| 100S | | | |
| Baseline | 11.7 | 4.1 | .73 |
| Post-Treatment | 3.34 | 2.9 | .47 |
| % Difference | −16% | −25% | −21% |
| 400S | | | |
| Baseline | .94 | 5.27 | .61 |
| Post-Treatment | .9 | 4.84 | .53 |
| % Difference | −4% | −20% | −3% |
| Gamma E only | | | |
| Baseline | .9 | 2.6 | .27 |
| Post-Treatment | .67 | 1.7 | .47 |
| % Difference | −25% | −90% | +83% |

While the sample sizes were too small to reach statistical significance, the differences were dramatic and clinically significant. The clinical trial designed by this inventor revealed that the sesame lignans were not more effective at the higher dose, but worked better at the 100 mg dose. While the group size of four completed subjects was small, the results with gamma-tocopherol alone were equivocal, as there were reductions in the Dityrosine and PLGF-1 levels, but Isoprostane levels increased by 83% of baseline mean. In addition, the decrease in the PLGF-1 level was due to a sharp decrease in one subject, from 0.9 to 0.2, while the remaining 3 subjects all had no change in PLGF-1 levels. Clearly, the sesame-gamma group had reduced levels of deep-tissue oxidation and a clinically significant reduction in the most important biomarker of cardiovascular disease, even in very healthy human volunteers.

The invention teaches a method of reducing lipid peroxidation and diminishing free radical damage in a human comprising the administration to said human of a therapeutically effective amount of a composition comprised of one or more natural tocopherols, synthetic tocopherol isomer, tocopherol analogue, or pharmaceutically acceptable salt or ester of a tocopherol, and one or more sesame lignans.

All of the ingredients used in the compositions of the present invention are obtainable commercially by suppliers well known to those skilled in the art of nutritional supplement formulation. Any dosage form may be employed for providing the patient with an effective dosage of the composition. The composition may be provided in a variety of dosage forms, such as a tablet, capsule, liquid, liposome, inhalant, sublingual tablet, suppository, oral spray dispersions, suspensions, solutions, capsules, transdermal delivery systems, dermal patch, etc., and may include a pharmaceutically acceptable carrier. Tablets and capsules represent the most advantageous oral dosage unit form. Any method known to those of ordinary skill in the art may be used to prepare capsules, tablets, or other dosage formulations. Pharmaceutically acceptable carriers include binding agents such as pregelatinized maize starch, polyvinylpryrrolidone or hydroxypropyl methycellulose; binders or fillers such as lactose, pentosan, microcrystalline cellulose or calcium hydrogen phosphate; lubricants such as magnesium stearate, talc or silica; disintegrants such as potato starch or sodium starch; or wetting agents such as sodium lauryl sulfate. Tablets or capsules can be coated by methods well known to those of ordinary skill in the art. Suitable carriers include, but are not limited to, sugars, starches, cellulose and derivatives thereof, wetting agents, lubricants such as sodium lauryl sulfate, stabilizers, tabletting agents, anti-oxidants, preservatives, coloring agents and flavoring agents. Reference may be made to REMINGTON'S PHARMACEUTICAL SCIENCES, (17th ed. 1985) for other carriers that would be suitable for combination with the present compositions. As will be appreciated, the pharmaceutical carriers used to prepare compositions in accordance with the present invention will depend on the administrable form to be used.

In a specific embodiment of the invention, the method is be used to treat a human suffering from one or more conditions that are related to lipid peroxidation such as atherosclerosis, premature aging, Alzheimer's disease, stroke, toxic hepatitis, viral hepatitis, peripheral vascular insufficiency, renal disease, and hyperglycemia. In an alternative embodiment, method is used in a healthy human who seeks to optimize his or her health.

In a specific embodiment of the invention, the administration of a composition comprised of gamma tocopherol and one or more sesame lignans in an amount sufficient to reduce lipid peroxidation and diminish free radical damage in a human results in reduced levels of dityrosine, a marker of lipid peroxidation. More specifically, blood dityrosine levels are reduced by about 10% to about 50% and in its most specific embodiment, blood dityrosine levels are reduced by about 25%.

In another specific embodiment of the invention, the administration of a composition comprised of gamma tocopherol and one or more sesame lignans in an amount sufficient to reduce lipid peroxidation and diminish free radical damage in a human results in reduced blood levels of isoprostane, another marker for lipid peroxidation, in a more specific embodiment, blood levels of isoprostane are reduced by about 10% to about 50%, and in its most specific embodiment, blood levels of isoprostane are reduced by about 21%.

In a yet another embodiment of the invention, the administration of a composition comprised of gamma tocopherol and one or more sesame lignans in an amount sufficient to reduce lipid peroxidation and diminish free radical damage in a human results in reduced blood levels of placental-like growth factor, the best marker for lipid peroxidation, more specifically, said blood levels of placental-like growth factor are reduced by about 8% to about 40%, and in the most specific embodiment, blood levels of placental-like growth factor are reduced by about 16%.

The method of the invention may be practiced using any natural or synthetic tocopherol, specifically alpha tocopherol or gamma tocopherol, and most specifically, gamma tocopherol. The sesame lignans used in the method of the invention may be any sesame lignan, more specifically one or more sesame lignans are selected from the group consisting of sesamin, episesamin, sesaminol, sesaminolinol, sesamol, sesamolin, sesaminol, sesamol dimer, sesaminol triglucoside, and sesaminol diglucoside. Most specifically, the sesame lignan used in the invention is episesamin.

The invention discloses the optimal doses to be used to achieve the reduction in lipid peroxidation that may be demonstrated by the markers of blood dityrosine, isoprostane, and PLGF-1. The optimal ranges are gamma tocopherol present in an amount of about 100 IU to about 2000 IU and one or more sesame lignans are present in an amount of about 10 mg to about 500 mg total. At this dosage range, the method is effective to reduce lipid peroxidation and diminish free radical damage in a human; reduce the oxidation of HDL and LDL by reducing lipid peroxidation and diminishing free radical damage in a human; increase levels of gamma tocopherol and diminish free radical damage in a human; reduce chylomicron oxidation and diminish free radical damage in a human.

In a more specific embodiment, gamma tocopherol is present in an amount of about 200 IU to about 1000 IU and one or more sesame lignans are present in an amount of about 20 mg to about 200 mg total. At this dosage range, the method is effective to reduce lipid peroxidation and diminish free radical damage in a human; reduce the oxidation of HDL and LDL by reducing lipid peroxidation and diminishing free radical damage to said human; increase levels of gamma tocopherol and diminish free radical damage in a human; reduce chylomicron oxidation and diminish free radical damage in a human.

Most specifically said gamma tocopherol is present in an amount of about 420 IU and one or more sesame lignans are present in an amount of about 100 mg total. At this specific dosage range the method is effective to reduce lipid peroxidation and diminish free radical damage to said human; reduce the oxidation of HDL and LDL by reducing lipid peroxidation and diminishing free radical damage to said human; increase levels of gamma tocopherol and diminish free radical damage to said human; reduce chylomicron oxidation and diminish free radical damage to said human.

The invention may also be employed as a method for treating atherosclerosis and its associated diseases including cardiovascular disorders, cerebrovascular disorders, peripheral vascular disorders, and intestinal vascular disorders in a human by administering to the human a therapeutically effective amount of a composition of one or more natural tocopherol, synthetic tocopherol isomer, analogue, or pharmaceutically acceptable salt or ester of a tocopherol, and one or more sesame lignans.

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the specific embodiments of the invention described specifically herein. Such equivalents are intended to be encompassed in the scope of the claims.

What is claimed is:

1. A method of reducing lipid peroxidation and diminishing free radical damage in a human comprising the administration to said human of a therapeutically effective amount of a composition comprised of one or more natural tocopherols, synthetic tocopherol isomer, tocopherol analogue, or pharmaceutically acceptable salt or ester of a tocopherol, and one or more sesame lignans.

2. The method of claim 1 wherein said composition is in a dosage form selected from the group consisting of a tablet, capsule, liquid, liposome, inhalant, sublingual tablet, suppository, oral spray and dermal patch.

3. The method of claim 1 wherein said composition is further comprised of pharmaceutically acceptable carrier.

4. The method according to claim 1, wherein said human suffers from one or more conditions selected from the group consisting of: atherosclerosis, premature aging, Alzheimer's disease, stroke, toxic hepatitis, viral hepatitis, peripheral vascular insufficiency, renal disease, and hyperglycemia.

5. The method according to claim 1, wherein the human is healthy and seeks to optimize his or her health.

6. The method according to claim 1, wherein said administration of a composition comprised of gamma tocopherol and one or more sesame lignans in an amount sufficient to reduce lipid peroxidation and diminish free radical damage to said human results in reduced levels of dityrosine in the blood of said human.

7. The method according to claim 6, wherein said reduced levels of dityrosine in the blood of said human are reduced by about 10% to about 50%.

8. The method according to claim 7, wherein said reduced levels of dityrosine in the blood of said human are reduced by about 25%.

9. The method according to claim 4, wherein said administration of a composition comprised of gamma tocopherol and one or more sesame lignans in an amount sufficient to reduce lipid peroxidation and diminish free radical damage to said human results in reduced levels of isoprostane in the blood of said human.

10. The method according to claim 9, wherein said reduced levels of isoprostane in the blood of said human are reduced by about 10% to about 50%.

11. The method according to claim 10, wherein said reduced levels of isoprostane in the blood of said human are reduced by about 21%.

12. The method according to claim 1, wherein said administration of a composition comprised of gamma tocopherol and one or more sesame lignans in an amount sufficient to reduce lipid peroxidation and diminish free radical damage to said human results in reduced levels of placental-like growth factor in the blood of said human.

13. The method according to claim 12, wherein said reduced levels of placental-like growth factor in the blood of said human are reduced by about 8% to about 40%.

14. The method according to claim 13, wherein said reduced levels of placental-like growth factor in the blood of said human are reduced by about 16%.

15. The method according to claim 1 wherein said natural or synthetic tocopherol is selected from the group consisting of alpha tocopherol and gamma tocopherol.

16. The method according to claim 15 wherein said natural or synthetic tocopherol is gamma tocopherol.

17. The method according to claim 1, wherein said one or more sesame lignans are selected from the group consisting of sesamin, episesamin, sesaminol, sesaminolinol, sesamol, sesamolin, sesaminol, sesamol dimer, sesaminol triglucoside, and sesaminol diglucoside.

18. The method according to claim 17, wherein said one or more sesame lignans is episesamin.

19. The method according to claim 16 wherein said natural or synthetic tocopherol is gamma tocopherol and is present in an amount of about 100 IU to about 2000 IU and said one or more sesame lignans are present in an amount of about 10 mg to about 500 mg total.

20. The method according to claim 16 wherein said composition comprising said gamma tocopherol present in an amount of about 100 IU to about 2000 IU and said one or more sesame lignans present in an amount of about 10 mg to about 500 mg total is effective to reduce lipid peroxidation and diminish free radical damage to said human.

21. The method according to claim 16 wherein said composition comprising said gamma tocopherol present in an amount of about 100 IU to about 2000 IU and said one or more sesame lignans present in an amount of about 10 mg to about 500 mg total is effective to reduce the oxidation of HDL and LDL by reducing lipid peroxidation and diminishing free radical damage to said human.

22. The method according to claim 16 wherein said composition comprising said gamma tocopherol present in an amount of about 100 IU to about 2000 IU and said one or more sesame lignans present in an amount of about 10 mg to about 500 mg total, or one of their pharmaceutically acceptable analogues or salts, is effective to increase levels of gamma tocopherol and diminish free radical damage to said human.

23. The method according to claim 16 wherein said composition comprising said gamma tocopherol present in an amount of about 100 IU to about 2000 IU and said one or more sesame lignans present in an amount of about 10 mg to about 500 mg total, is effective to reduce chylomicron oxidation and diminish free radical damage to said human.

24. The method according to claim 16, wherein said gamma tocopherol is present in an amount of about 200 IU to about 1000 IU and said one or more sesame lignans are present in an amount of about 20 mg to about 200 mg total.

25. The method according to claim 24 wherein said composition comprising said gamma tocopherol present in an amount of about 200 IU to about 1000 IU and said one or more sesame lignans present in an amount of about 20 mg to about 200 mg total, is effective to reduce lipid peroxidation and diminish free radical damage to said human.

26. The method according to claim 24 wherein said composition comprising said gamma tocopherol present in an amount of about 200 IU to about 1000 IU and said one or more sesame lignans present in an amount of about 20 mg to about 200 mg total, is effective to reduce the oxidation of HDL and LDL by reducing lipid peroxidation.

27. The method according to claim 24 wherein said composition comprising said gamma tocopherol present in an amount of about 200 IU to about 1000 IU and said one or more sesame lignans present in an amount of about 20 mg to about 200 mg total, is effective to increase levels of gamma tocopherol and diminish free radical damage to said human.

28. The method according to claim 24 wherein said composition comprising said gamma tocopherol present in an amount of about 200 IU to about 1000 IU and said one or more sesame lignans present in an amount of about 20 mg to about 200 mg total, is effective to reduce chylomicron oxidation and diminish free radical damage to said human.

29. The method according to claim 24, wherein said gamma tocopherol is present in an amount of about 420 IU and said one or more sesame lignans are present in an amount of about 100 mg total.

30. The method according to claim 29 wherein said composition comprising said gamma tocopherol present in an amount of about 420 IU and said one or more sesame lignans present in an amount of about 100 mg total, is effective to reduce lipid peroxidation and diminish free radical damage to said human.

31. The method according to claim 29 wherein said composition comprising said gamma tocopherol present in an amount of about 420 IU and said one or more sesame lignans present in an amount of about 100 mg total, is effective to reduce the oxidation of HDL and LDL by reducing lipid peroxidation.

32. The method according to claim 29 wherein said composition comprising said gamma tocopherol present in an amount of about 420 IU and said one or more sesame lignans present in an amount of about 100 mg total, is effective to increase levels of gamma tocopherol and diminish free radical damage to said human.

33. The method according to claim 29 wherein said composition comprising said gamma tocopherol present in an amount of about 420 IU and said one or more sesame lignans present in an amount of about 100 mg total, is effective to reduce chylomicron oxidation and diminish free radical damage to said human.

34. A method of treating atherosclerosis and its associated diseases including cardiovascular disorders, cerebrovascular disorders, peripheral vascular disorders, and intestinal vascular disorders in a human comprising the administration to said human of a therapeutically effective amount of a composition comprising one or more natural tocopherol, synthetic tocopherol isomer, analogue, or pharmaceutically acceptable salt or ester of a tocopherol, and one or more sesame lignans.
